(19) United States
(12) Patent Application Publication
(10) Pub. No.: US 2020/0061009 A1
(43) Pub. Date: Feb. 27, 2020

(54) Health functional food for reducing and preventing capillary hemorrhage

(71) Applicant: STARLINGFORCE CO., LTD., Seoul (KR)

(72) Inventor: Dae Kil JANG, Gwangmyeong-si (KR)

(21) Appl. No.: 16/138,314

(22) Filed: Sep. 21, 2018

(30) Foreign Application Priority Data
Aug. 27, 2018 (KR) 10-2018-0100468

(51) Int. Cl. A61K 31/198; A61K 31/191; A61K 31/25; A61K 31/4406; A61K 31/07; A61K 31/555; A61K 31/675; A61K 31/341; A61K 31/355; A61K 33/08; A61K 31/20; A61K 31/519; A61K 31/045; A61K 31/047; A61K 33/00; A61K 33/24; A61K 31/717; A61K 31/185; A61K 31/194; A61K 36/286; A61K 36/076; A61K 36/42; A61K 36/65; A61K 36/605; A61K 36/232; A61K 36/744; A23L 33/175; A23L 33/16; A23L 33/125; A23L 33/105; A23L 33/155

(52) U.S. Cl. CPC A61K 31/198; A23V 2002/00; A61K 31/25; A61K 31/4406; A61K 31/07; A61K 31/555; A61K 31/675; A61K 31/341; A61K 31/355; A61K 33/08; A61K 31/20; A61K 31/519; A61K 31/045; A61K 31/047; A61K 33/00; A61K 33/24; A61K 31/717; A61K 31/185; A61K 31/194; A61K 36/286; A61K 36/076; A61K 36/42; A61K 36/65; A61K 36/605; A61K 36/232; A61K 36/744; A23L 33/175; A23L 33/16; A23L 33/125; A23L 33/105; A23L 33/155; A61K 31/191

(57) ABSTRACT

Provided is a health functional food that contains a variety of active ingredients and can improve blood flow as well as reduce or prevent capillary hemorrhage, which can help to maintain health.

### BACKGROUND OF THE INVENTION

### Field of the Invention

The present invention relates to a health functional food that contains a variety of active ingredients and can improve blood flow as well as reduce or prevent capillary hemorrhage, to help maintain health.

### Description of the Related Art

In recent years, changes in living environments such as westernization of diet, lack of exercise and excessive stress have brought about an increase in diseases relating to blood or circulatory systems, such as arteriosclerosis and cerebral infarction, and diseases adversely affecting blood circulation, such as hyperlipidemia and diabetes. These diseases are considered to cause a deterioration in blood flow of microvasculature and capillary blood vessels, which may have various negative effects on the human body. In addition, it has been reported that blood flow is also associated with itching of the skin, fatigue, hypertension, and the like.

In general, blood circulation, that is, blood flow, refers to a phenomenon in which blood circulates throughout the body through blood vessels, based on the heart, which functions to supply oxygen to respective tissues and release carbon dioxide, to distribute nutrients and release metabolic products, to transport hormones from various endocrine glands and regulate functions of specific organs, and to control pathogens and regulate body temperature, osmotic pressure and moisture. Blood vessels are composed of arteries, capillary blood vessels (capillaries) and veins. Each artery is composed of the tunica adventitia including the external elastic lamina and connective tissue, the tunica media including many smooth muscles and a large number of elastic fibers therein, and the tunica intima including the innermost endothelium, connective tissue surrounding the endothelium and the internal elastic lamina composed of a bundle of elastic fibers. Capillary blood vessels (capillaries) are thin vessels which connect arterioles to veinlets, are finely branched in the form of a net, have a small diameter of 0.008 to 0.02 mm to allow erythrocytes to pass therethrough, and are invisible to the naked eye. These capillaries transport oxygen and nutrients in the blood to respective tissues, and send carbon dioxide (CO₂) and waste produced in the tissues to blood vessels.

The tunica adventitia of the vein is relatively thick, while the tunica media and tunica intima thereof are thin. The vein has no pressure and has a weaker blood flow than other blood vessels. For this reason, the vein has a valve to prevent backflow since there is a risk that blood may backflow from the veins below the heart.

In addition, a blood circulation disorder refers to a phenomenon in which blood vessels that extend to every corner of the human body lose elasticity and cholesterol is thus deposited on inner walls thereof, which causes the lumen of the blood vessels to narrow and thus results in unfavorable blood circulation. The blood circulation disorder induces acrohypothermy, numbness, pain in the back of neck, shoulder discomfort, memory loss, lethargy, weakening of concentration, dizziness and chronic fatigue symptoms which make it very difficult to lead a normal life.

Cardiovascular diseases, such as hypertension, arteriosclerosis, heart disease and stroke, caused by the blood circulation disorder, are currently dominant adult diseases. Cancer is a disease with the highest mortality rate at or before the age of 70, but cardiovascular disease is the primary death cause after the age of 70. These cardiovascular diseases are closely related to one another. As mentioned above, in accordance with rapid economic development, the development of dietary life, changes in social environments and westernization, the disease trend has been gradually westernized and cardiovascular diseases are gradually increased. Increases in total calorie intake and fat intake cause a significant increase in blood lipid levels, including blood cholesterol level, and a significant increase in the incidence of atherosclerotic cardiovascular diseases such as coronary artery diseases, stroke and peripheral vascular diseases. The causes of cardiovascular diseases such as hyperlipidemia, atherosclerosis, myocardial infarction and cerebral thrombosis are well-known to be associated with ingestion of processed foods with high fat contents and low dietary fiber contents, genetic factors, smoking, drinking, hypertension, diabetes, obesity, lack of exercise and excessive stress. The basic lesions that cause these cardiovascular diseases are atherosclerosis and a variety of risk factors have been found by epidemiological and experimental studies on the cause of atherosclerosis. Pathogenesis of these lesions is as follows: physical or chemical wounds cause damage to the surface of monolayer cells of blood vessels, thrombus-adhesion receptors capable of inducing formation of thrombus on the blood vessel walls are expressed, and monocytes, T-lymphocytes and platelets adhere thereto and permeate into the blood vessel walls through tunica intima cells of the blood vessels. At this time, lipid proteins in blood are also incorporated therein. The monocytes incorporated into the blood vessel walls digest lipid proteins, which are then converted into macrophages. The macrophages allow cholesterol to be accumulated to form cells. The form cells destroy or thicken the blood vessel walls through repeated inflammatory reactions and cell growth, which causes platelets in the blood to adhere thereto, forming blood clots (thrombosis). This process eventually leads to sudden blockage of blood supply to the heart because of stenosis of blood vessels derived from arteriosclerosis and complete closure of blood vessels derived from blood clots, which eventually induces death due to myocardial infarction.

In particular, blood flow is affected by flowability of blood, flexibility of erythrocytes or leukocytes and the coagulation ability of platelets or the like due to hyperglycemia. Specifically, hemocytes such as erythrocytes and leukocytes are known to account for about 40% of the blood volume, and affect blood flowability, particularly in microvasculature. When deterioration in such blood flow is continued for a long period of time, for example, a phenomenon called “thrombosis”, in which blood clots readily obstruct erythrocytes or leukocytes in the microvasculature wherein the flexibility of the blood vessel is lost and flexibility of erythrocytes is deteriorated, readily occurs. This is one of causes of circulatory diseases. In the case of poisoning, the flow of blood is ceased and tissue necrosis occurs in the surrounding environment. Thus, easy flow of blood in the bio-systems has a key role in maintaining health.

Therefore, a number of food and food ingredients that have the potential to improve blood flow have been developed and reported. For example, foods such as dark vinegar, plum pickles and the like can be mentioned as familiar food materials. In addition, it was reported in Japanese Patent Application Publication Hei 7-138168 that a fish bile extract in a polar solvent improves blood flowability. Also, Japanese Patent Application Publication No. 2002-97143 reports that glucosamine salts or glucosamine derivatives prevent blood clots or improve blood flow. However, improvement of blood flow is mainly due to the action associated with flowability of blood, and foods or pharmaceutical compositions containing ingredients having effects of improving blood flow as well as strength or elasticity of blood vessels, that is, having an effect of protecting blood vessels, have not been provided. On the other hand, Japanese Patent Application Laid-Open No. 2000-135071 discloses that a wine extract has better blood vessel strengthening activity than a polyphenol-containing extract. However, there has been no study on the flowability of blood.

Therefore, in a true sense, there is demand for a composition with improved blood flow that is highly effective in improving blood flow in vivo, that is, enhancing blood flowability and in protecting blood vessels.

Meanwhile, the present inventors have developed a health functional food for improving blood flow, as disclosed in Korean Patent No. 10-1424335 in relation to improvement in blood flow. Also, the present inventors developed a health functional food that can prevent hemorrhage from injured capillary walls, in addition to improving blood flow.

### PRIOR ART

### Patent Document

(Patent Document 0001) Korean Patent No. 10-1424335

### SUMMARY OF THE INVENTION

Therefore, the present invention has been made in view of the above problems, and it is an object of the present invention to provide a health functional food that can prevent capillary hemorrhage by curing and repairing the walls of capillaries when the capillaries are injured.

The objects to be accomplished by the present invention are not limited to those mentioned above and other objects will be clearly understood to those skilled in the art from the following description.

In accordance with the present invention, provided is a health functional food for reducing or preventing capillary hemorrhage including 40 to 50 parts by weight of L-glutamine, to 50 parts by weight of L-lysine hydrochloride, 5 to 15 parts by weight of L-arginine, 10 to 20 parts by weight of N-acetyl glucosamine, 10 to 20 parts by weight of a safflower seed extract powder, 0.1 to 5 parts by weight of a cinnamon extract powder, 150 to 200 parts by weight of a white poria cocos Wolf extract powder, 1 to 3 parts by weight of copper gluconate, 1 to 3 parts by weight of glycerin fatty acid ester, 2 to 4 parts by weight of nicotinamide, 4 to 6 parts by weight of a pumpkin extract powder, 50 to 70 parts by weight of a Paeonia japonica extract powder, 1 to 2 parts by weight of vitamin A, 0.1 to 0.5 parts by weight of vitamin B12, 15 to 25 parts by weight of vitamin B1, 0.2 to 0.6 parts by weight of vitamin B6, 100 to 200 parts by weight of vitamin C, 5 to 15 parts by weight of vitamin E, 10 to 20 parts by weight of magnesium oxide, 15 to 25 parts by weight of a mulberry leaf extract powder, 10 to 15 parts by weight of magnesium stearate, 0.1 to 0.3 parts by weight of folic acid, 3 to 7 parts by weight of octacosanol, 100 to 150 parts by weight of an Angelica gigas extract powder, 5 to 10 parts by weight of a Cordyceps sinensis extract powder, 20 to 30 parts by weight of inositol, 10 to 15 parts by weight of silicon dioxide, 5 to 10 parts by weight of titanium dioxide, 1 to 3 parts by weight of Gardenia yellow color, 5 to 15 parts by weight of carboxymethyl cellulose calcium, 5 to 10 parts by weight of quercetin, 5 to 10 parts by weight of a coenzyme, 50 to 80 parts by weight of taurine, 50 to 80 parts by weight of a kudzu extract powder, 10 to 30 parts by weight of a pine bark extract powder, 0.1 to 1.5 parts by weight of ferrous fumarate, 15 to 25 parts by weight of hydroxypropyl methylcellulose (HPMC), and 15 to 25 parts by weight of hydroxypropyl cellulose (HPC).

According to the present invention, the cinnamon extract powder, the white poria cocos Wolf extract powder, the pumpkin extract powder, the Paeonia japonica extract powder, the mulberry leaf extract powder, the Angelica gigas extract powder, the Cordyceps sinensis extract powder, the kudzu extract powder, and the pine bark extract powder are obtained by hot water extraction. For example, a white poria cocos Wolf is extracted in 3 to 10-times amount of water by hot water extraction using an extractor equipped with a reflux condenser for every 6 hours three times, the extract is collected and concentrated under reduced pressure, and the resulting extract is then lyophilized and produced into a powder, which is used in the present invention. The method of obtaining the powder is not limited to hot water extraction and the powder can be obtained by solvent extraction using alcohol or the like, as a well-known method.

### BRIEF DESCRIPTION OF THE DRAWINGS

The above and other objects, features and other advantages of the present invention will be more clearly understood from the following detailed description taken in conjunction with the accompanying drawings, in which:

FIGS. 1A to 1E are images showing changes in capillaries in test subjects with capillary hemorrhage after administration of two 1,000 mg tablets obtained from food prepared in accordance with Example 1; and

FIGS. 2A to 2C are images showing changes in capillaries in test subjects with capillary hemorrhage after administration of two 1,000 mg tablets obtained from food prepared in accordance with Example 2.

### DETAILED DESCRIPTION OF THE INVENTION

Hereinafter, preferred examples of the present invention will be described in more detail. However, these examples are provided only for illustration of the present invention and should not construed as limiting the scope of the present invention.

### Example 1

In Example 1, a health functional food was prepared in accordance with the following ingredients and compositions.

L-glutamine 45.5 mg, L-lysine hydrochloride 45.5 mg, L-arginine (Korea, Daesang Corp.) 10 mg, N-acetyl glucosamine 14 mg, safflower seed extract powder 14 mg, cinnamon extract powder mg, white poria cocos Wolf extract powder 170 mg, copper gluconate 1.917 mg, glycerin fatty acid ester 2.1 mg, nicotinamide (China) 3.2 mg, pumpkin extract powder 5 mg, Paeonia japonica extract powder 60 mg, vitamin A mixture powder (Switzerland) 1.504 mg, vitamin B12 (100%) 0.333 mg, vitamin B1 nitrate (Germany) 20.5 mg, vitamin B6 hydrochloride (Germany) 0.4 mg, vitamin C (United Kingdom) 150 mg, vitamin E mixture powder (DSM) 11.2 mg, magnesium oxide 17 mg, mulberry (leaf) extract powder 21.8 mg, magnesium stearate (Singapore) 12.6 mg, folic acid (Switzerland) 0.166 mg, octacosanol (1%, Korea) 5 mg, Angelica gigas extract powder 110 mg, Cordyceps sinensis extract powder 8.3 mg, inositol 24.5 mg, silicon dioxide 12.6 mg, titanium dioxide 7 mg, Gardenia yellow color (600 PD, Dube Corp. Inc.) 2 mg, carboxymethyl cellulose calcium 10 mg, quercetin 7 mg, coenzyme Q10 (Jupiter International Co., Ltd.) 8.4 mg, taurine 69.5 mg, kudzu (root) extract powder 69.5 mg, pine bark extract powder 16.4 mg, ferrous fumarate 1.08 mg, hydroxypropyl methylcellulose (HPMC) 21 mg, and hydroxypropyl cellulose (HPC) 20 mg.

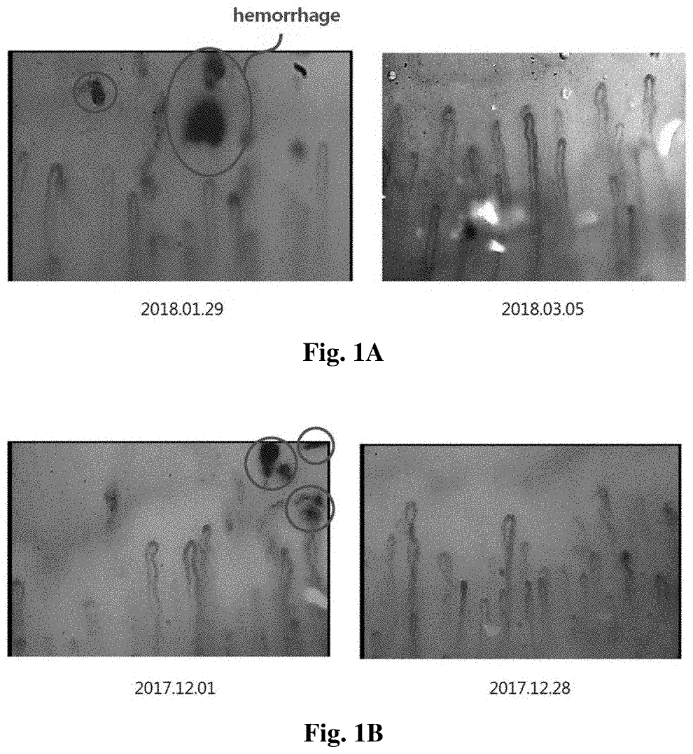

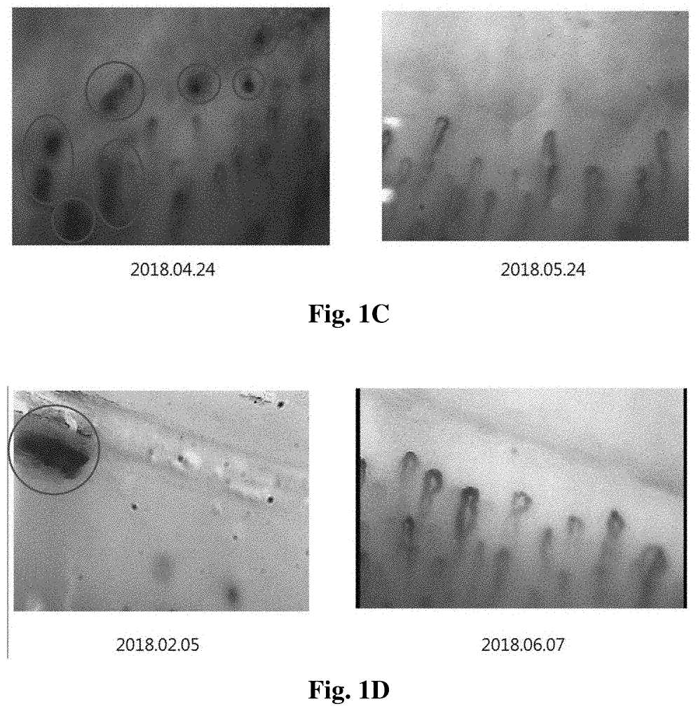

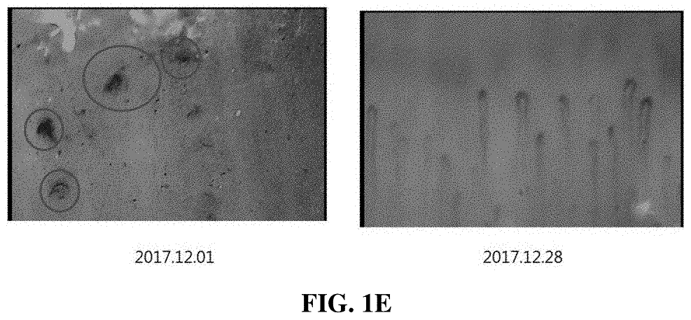

FIGS. 1A to 1E are images showing changes in capillaries in test subjects with capillary hemorrhage after administration of two 1,000 mg tablets obtained from food prepared in accordance with Example 1.

TABLE 1

| | | Administration | Comparison in images between |
|---|---|---|---|
| Name | Gender | period | before and after administration |
| Doo*Hee | Male | 35 days | As can be seen from FIG. 1A, |
| | | (2018.01.29. to | hemorrhage disappeared in the |
| | | 2018.03.05.) | central upper part after |
| | | | administration of food of |
| | | | Example 1 |
| Park*Soon | Female | 27 days | As can be seen from FIG. 1B, |
| | | (2017.12.01. to | hemorrhage disappeared in the |
| | | 2017.12.28.) | right upper part after |
| | | | administration of food of |
| | | | Example 1 |
| Yoon*Ha | Male | 30 days | As can be seen from FIG. 1C, |
| | | (2018.04.24. to | hemorrhage like small spots |
| | | 018.05.24.) | disappeared and capillary wall |
| | | | became bright |
| Lee*Hwa | Female | 4 months | As can be seen from FIG. 1D, |
| | | (2018.02.05. to | hemorrhage disappeared in the left |
| | | 2018.06.07.) | upper part after administration of |
| | | | food of Example 1 |
| Joo*Ok | Female | 27 days | As can be seen from FIG. 1E, |
| | | (2017.12.01. to | overall scattered small hemorrhage |
| | | 2017.12.28.) | almost disappeared after |
| | | | f administration of ood of |
| | | | Example 1 |

As can be seen from FIGS. 1A to 1E and Table 1, when the health functional food of Example 1 according to the present invention is administrated, capillary hemorrhage can be reduced.

### Example 2

In Example 2, a health functional food was prepared in accordance with the following ingredients and compositions.

L-glutamine 45.5 mg, L-lysine hydrochloride 45.5 mg, L-arginine (Korea, Daesang Corp.) 10 mg, N-acetyl glucosamine 14 mg, safflower seed extract powder 14 mg, cinnamon extract powder 1 mg, white poria cocos Wolf extract powder 110 mg, fermented Hosta longipes leaf powder 60 mg, copper gluconate 1.917 mg, glycerin fatty acid ester 2.1 mg, nicotinamide (China) 3.2 mg, pumpkin extract powder 5 mg, Paeonia japonica extract powder 60 mg, vitamin A mixture powder (Switzerland) 1.504 mg, vitamin B12 (100%) 0.333 mg, vitamin B1 nitrate (Germany) 20.5 mg, vitamin B6 hydrochloride (Germany) 0.4 mg, vitamin C (United Kingdom) 135 mg, vitamin E mixture powder (DSM) 11.2 mg, magnesium oxide 17 mg, mulberry (leaf) extract powder 21.8 mg, fermented Scabiosa mansenensis powder 15 mg, magnesium stearate (Singapore) 12.6 mg, folic acid (Switzerland) 0.166 mg, octacosanol (1%, Korea) 5 mg, Angelica gigas extract powder 110 mg, Cordyceps sinensis extract powder 8.3 mg, inositol 24.5 mg, silicon dioxide 12.6 mg, titanium dioxide 7 mg, Gardenia yellow color (600PD, Dube Corp. Inc.) 2 mg, carboxymethyl cellulose calcium 10 mg, quercetin 7 mg, coenzyme Q10 (Jupiter International Co., Ltd.) 8.4 mg, taurine 69.5 mg, kudzu (root) extract powder 69.5 mg, pine bark extract powder 16.4 mg, ferrous fumarate 1.08 mg, hydroxypropyl methylcellulose (HPMC) 21 mg, and hydroxypropyl cellulose (HPC) 20 mg.

Here, the fermented powder was obtained by crushing dried Hosta longipes leaves (or Scabiosa mansenensis), immersing the same in distilled water, seeding a Bacillus subtilis starter, fermenting at 41° C. for 5 days to obtain a fermented product, and lyophilizing and grinding the fermented product.

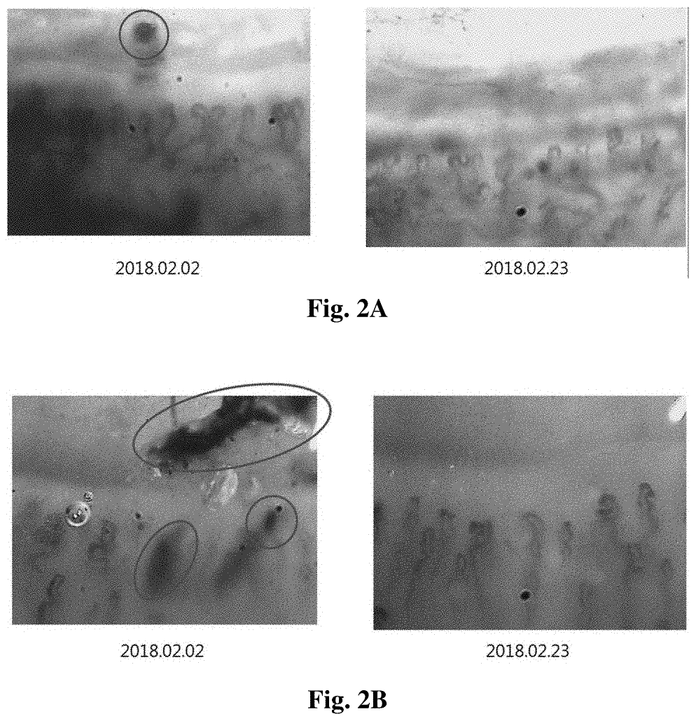

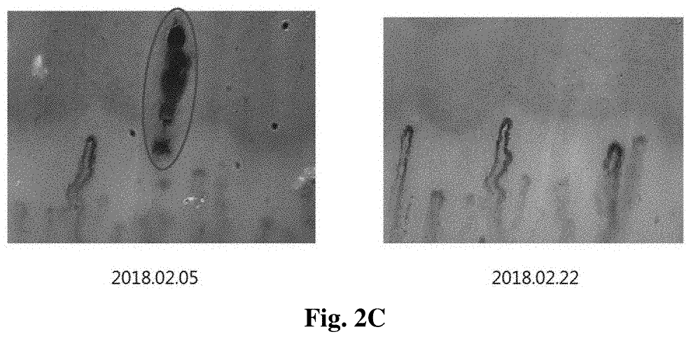

FIGS. 2A to 2C are images showing changes in capillaries in test subjects with capillary hemorrhage after administration of two 1,000 mg tablets obtained from food prepared in accordance with Example 2.

TABLE 2

| | | Administration | Comparison in images between |
|---|---|---|---|
| Name | Gender | period | before and after administration |
| Kim*Sook | Female | 3 weeks | As can be seen from FIG. 2A, |
| | | (2018.02.02. to | hemorrhage disappeared in the |
| | | 2018.02.23.) | central upper part and capillary |
| | | | became entirely bright after |
| | | | administration of food of |
| | | | Example 2 |
| An*Ja | Female | 3 weeks | As can be seen from FIG. 2B, |
| | | (2018.02.02. to | great hemorrhage disappeared in |
| | | 2018.02.23.) | the right upper edge after |
| | | | administration of food of |
| | | | Example 2 |
| Kim*Ja | Female | 17 days | As can be seen from FIG. 2C, |
| | | (2018.02.05. to | great hemorrhage completely |
| | | 2018.02.22.) | disappeared in the central part after |
| | | | administration of food of |
| | | | Example 2 |

As can be seen from FIGS. 2A to 2E and Table 2, when the health functional food of Example 2 according to the present invention is administrated, capillary hemorrhage can be reduced. In addition, Example 2 is different from Example 1 in that Example 2 exhibits an effect of reducing hemorrhage even if the food is administered for a relatively short period. More specifically, Example 1 exhibits an effect of reducing hemorrhage after administration for a period of 30 days or more on average, Example 2 exhibits an effect of reducing hemorrhage after administration for a period of about 20 days on average.

The health functional food for reducing or preventing capillary hemorrhage according to the present invention cures wounds in capillaries, thereby reducing or preventing hemorrhage, and in the long term, maximizing strength and elasticity of blood vessels, and preventing and treating blood circulation disorders.

As is apparent from the above description, the present invention provides a health functional food that contains a variety of active ingredients and can improve blood flow as well as reduce or prevent capillary hemorrhage, which is advantageously beneficial for maintaining health.

The effects of the present invention are not limited to those mentioned above and other effects will be clearly understood to those skilled in the art from the aforementioned description.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A health functional food for reducing or preventing capillary hemorrhage, comprising:
40 to 50 parts by weight of L-glutamine;
40 to 50 parts by weight of L-lysine hydrochloride;
5 to 15 parts by weight of L-arginine;
10 to 20 parts by weight of N-acetyl glucosamine;
10 to 20 parts by weight of a safflower seed extract powder;
0.1 to 5 parts by weight of a cinnamon extract powder;
150 to 200 parts by weight of a white poria cocos Wolf extract powder;
1 to 3 parts by weight of copper gluconate;
1 to 3 parts by weight of glycerin fatty acid ester;
2 to 4 parts by weight of nicotinamide;
4 to 6 parts by weight of a pumpkin extract powder;
50 to 70 parts by weight of a Paeonia japonica extract powder;
1 to 2 parts by weight of vitamin A;
0.1 to 0.5 parts by weight of vitamin B12;
15 to 25 parts by weight of vitamin B1;
0.2 to 0.6 parts by weight of vitamin B6;
100 to 200 parts by weight of vitamin C;
5 to 15 parts by weight of vitamin E;
10 to 20 parts by weight of magnesium oxide;
15 to 25 parts by weight of a mulberry leaf extract powder;
10 to 15 parts by weight of magnesium stearate;
0.1 to 0.3 parts by weight of folic acid;
3 to 7 parts by weight of octacosanol;
100 to 150 parts by weight of an Angelica gigas extract powder;
5 to 10 parts by weight of a Cordyceps sinensis extract powder;
20 to 30 parts by weight of inositol;
10 to 15 parts by weight of silicon dioxide;
5 to 10 parts by weight of titanium dioxide;
1 to 3 parts by weight of Gardenia yellow color;
5 to 15 parts by weight of carboxymethyl cellulose calcium;
5 to 10 parts by weight of quercetin;
5 to 10 parts by weight of a coenzyme;
50 to 80 parts by weight of taurine;
50 to 80 parts by weight of a kudzu extract powder;
10 to 30 parts by weight of a pine bark extract powder;
0.1 to 1.5 parts by weight of ferrous fumarate;
15 to 25 parts by weight of hydroxypropyl methylcellulose (HPMC); and
to 25 parts by weight of hydroxypropyl cellulose (HPC).
